(19) United States
(12) Patent Application Publication
(10) Pub. No.: US 2010/0239666 A1
(43) Pub. Date: Sep. 23, 2010

(54) Process for producing mouldings from (meth) acrylate copolymers by means of injection moulding

(75) Inventors: Hans-Ulrich Petereit, Darmstadt (DE); Thomas Beckert, Warthausen (DE); Manfred Assmus, Bickenbach (DE); Wemer Hoess, Heusenstamm (DE); Wolfgang Fuchs, Alsbach (DE); Hartmut Schikowsky, Darmstadt (DE)

Correspondence Address: GLAXOSMITHKLINE, Global Patents-UW2220, P.O.Box 1539, King of Prussia, PA, 19406-0939, US

(73) Assignee: GLAXO GROUP LIMITED

(21) Appl. No.: 12/793,549

(22) Filed: Jun. 3, 2010

Related U.S. Application Data
(63) Continuation of application No. 10/333,930, filed on Jun. 27, 2003.

(30) Foreign Application Priority Data
Jun. 5, 2001 (DE) 101 27 134.4

(51) Int. Cl. A61K 9/52; A61K 47/32; A61K 31/60; A61K 31/34; A61K 31/35; A61K 31/40; A61K 31/44; A61K 31/43; A61K 31/135; A61K 31/496; A61K 31/52; A61K 38/22; A61K 31/4525; A61K 31/12; A61K 31/439; A61K 31/4164; A61K 31/519; A61K 31/5415; A61K 31/277; A61K 31/16; A61K 31/4353; A61P 43/00; A61P 5/00

(52) U.S. Cl. 424/457; 514/772.4; 514/159; 514/471; 514/460; 514/423; 514/356; 514/197; 514/657; 514/253.07; 514/263.1; 514/9.7; 514/321; 514/682; 514/305; 514/400; 514/196; 514/249; 514/223.2; 514/523; 514/629; 514/282; 514/283

(57) ABSTRACT

The invention relates to a process for producing mouldings by injection moulding the steps in the process being a) melting and mixing of a (meth)acrylate copolymer composed of from 85 to 98% by weight of C1-C4-alkyl (meth)acrylates capable of free-radical polymerization and from 15 to 2% by weight of (meth)acrylate monomers having a quaternary ammonium group in the alkyl radical, with from 10 to 25% by weight of a plasticizer, and also from 10 to 50% by weight of a dryers [sic] and/or from 0.1 to 3% by weight of a release agent, and, where appropriate, with other conventional pharmaceutical additives or auxiliaries and/or with an active pharmaceutical ingredient, b) devolatilizing the mixture at temperatures of at least 120° C., thus reducing the content of the low-boiling constituents with a vapour pressure of at least 1.9 bar at 120° C. to not more than 0.5% by weight, and c) injecting the devolatilized mixture at a temperature of from 80 to 160° C. into the mould of an injection moulding system and removing the resultant moulding from the mould.

The invention relates to a process for producing mouldings by means of injection moulding, and to the mouldings themselves.

### PRIOR ART

(Meth)acrylate copolymers which contain monomers having quaternary ammonium groups, e.g. trimethylammonium-methlymethacrylate [sic] chloride and their use for delayed-release pharmaceutical coatings have been known for a long time (e.g. from EP-A 181 515 or DE-C 1 617 751). Processing takes place in organic solution or in the form of an aqueous dispersion, e.g. by spraying onto pharmaceutical cores, or else without solvent in the presence of flow aids by application in the melt (see EP-A 0 727 205).

EP 0 704 207 A2 describes thermoplastics for drug coverings soluble in intestinal fluid. These are copolymers made from 16-40% by weight of acrylic or methacrylic acid, from 30 to 80% by weight of methyl acrylate, and from 0 to 40% by weight of other alkyl (meth)acrylates.

In the example, appropriate copolymers are melted at 160° C. and mixed after addition of 6% by weight of glycerol monostearate. The mixture is broken and ground to give a powder. The powder is charged to the antechamber of an injection mould and injected at 170° C. under a pressure of 150 bar through an aperture of width 0.5 mm into the mould cavity. Cooling gives bubble-free, slightly opaque, thin-walled pharmaceutical capsules. No particular measures are disclosed for removing low-boiling constituents immediately prior to injection moulding.

### OBJECT AND ACHIEVEMENT OF OBJECT

It was an object to provide a process which permits the known (meth)acrylate copolymers containing monomers having quaternary ammonium groups to be processed by injection moulding. The intention is that the resultant mouldings have delayed-release properties and meet high mechanical requirements and therefore can be used, for example, as capsules (hard capsules) which serve as containers for pelleted active pharmaceutical ingredients.

The object is achieved by means of a

process for producing mouldings by injection moulding, the steps in the process being
a) melting and mixing of a (meth)acrylate copolymer composed of from 85 to 98% by weight of C1-C4-alkyl (meth)acrylates capable of free-radical polymerization and from 15 to 2% by weight of (meth)acrylate monomers having a quaternary ammonium group in the alkyl radical, with from 10 to 25% by weight of a plasticizer, and also from 10 to 50% by weight of a dryers [sic] and/or from 0.1 to 3% by weight of a release agent, and, where appropriate, with other conventional pharmaceutical additives or auxiliaries and/or with one or more active pharmaceutical ingredients,
b) devolatilizing the mixture at temperatures of at least 120° C., thus reducing the content of the low-boiling constituents with a vapour pressure of at least 1.9 bar at 120° C. to not more than 0.5% by weight, and
c) injecting the devolatilized mixture at a temperature of from 80 to 160° C. into the mould of an injection moulding system and removing the resultant moulding from the mould.

Novel injection mouldings which meet high mechanical requirements are obtainable by means of the process of the invention.

### WORKING OF THE INVENTION

The process of the invention for producing mouldings by means of injection moulding divides into steps a), b) and c) of the process.

### Step a) of the Process

Melting and mixing of a (meth)acrylate copolymer composed of from 85 to 98% by weight of C1-C4-alkyl (meth)acrylates capable of free-radical polymerization and from 15 to 2% by weight of (meth)acrylate monomers having a quaternary ammonium group in the alkyl radical, with from 10 to 25% by weight of a plasticizer, and also from 10 to 50% by weight of a dryers [sic] and/or from 0.1 to 3% by weight of a release agent, and, where appropriate, with other conventional pharmaceutical additives or auxiliaries and/or with one or more active pharmaceutical ingredients, [sic]

The % by weight data here are based in each case on the (meth)acrylate copolymer. The (meth)acrylate copolymer, which is in pellet or powder form, is preferably melted in an extruder at a temperature of from 70 to 140° C. Dryers and/or release agents and the plasticizer may be incorporated here simultaneously or in succession, in any desired sequence. This also applies to any other conventional pharmaceutical auxiliaries or additives present, and to any active pharmaceutical ingredient present.

### The (Meth)Acrylate Copolymer

Examples of appropriate (meth)acrylate copolymers are known from EP-A 181 515 or DE-C 1 617 751. These are polymers with pH-independent solubility or swellability and are suitable for pharmaceutical coatings. A possible preparation process which may be mentioned is bulk polymerization in the presence of a free-radical-generating initiator dissolved in the monomer mixture. The polymer may also be prepared by means of solution or precipitation polymerization. The polymer can thus be obtained in the form of a fine powder, and in the case of bulk polymerization this is obtainable by grinding, and in the case of solution or precipitation polymerization by spray drying, for example.

The (meth)acrylate copolymer is composed of from 85 to 98% by weight of C1-C4-alkyl (meth)acrylates capable of free-radical polymerization and of from 15 to 2% by weight of (meth)acrylate monomers having a quaternary ammonium group in the alkyl radical.

Preferred C1-C4-alkyl (meth)acrylates are methyl acrylate, ethyl acrylate, butyl acrylate, butyl methacrylate and methyl methacrylate.

A particularly preferred (meth)acrylate monomer having quaternary ammonium groups is 2-trimethylammoniumethyl methacrylate chloride.

An example of an appropriate copolymer may have a structure made from 50-70% by weight of methyl methacrylate, from 20 to 40% by weight of ethyl acrylate and from 7 to 2% by weight of 2-trimethylammoniumethyl methacrylate chloride.

A specific suitable copolymer contains to have [sic] a structure made from 65% by weight of methyl methacrylate, 30% by weight of ethyl acrylate and 5% by weight of 2-trimethylammoniumethyl methacrylate chloride (EUDRAGIT® RS).

Another suitable (meth)acrylate copolymer may, for example, have a structure made from 85-less than 93% by weight of C1-C4-alkyl (meth)acrylates and from more than 7 to 15% by weight of (meth)acrylate monomers having a quaternary ammonium group in the alkyl radical. (Meth)acrylate monomers of this type are commercially available and have long been used for delayed-release coatings.

A specific suitable copolymer contains, for example, 60% by weight of methyl methacrylate, 30% by weight of ethyl acrylate and 10% by weight of 2-trimethyl-ammoniumethly [sic] methacrylate chloride (EUDRAGIT® RL).

### Mixtures

The meth)acrylate [sic] copolymer is present in a mixture with a plasticizer and either with a dryer and/or [sic] with a release agent. In a manner known per se, there may also be other conventional pharmaceutical auxiliaries and/or an active pharmaceutical ingredient present.

The addition of plasticizer reduces the brittleness of the mouldings. The result is a reduction in the proportion of broken mouldings after demoulding. Without plasticizer, the proportion of mouldings satisfactorily removed is generally about 85% for most mixtures. With plasticizer addition, the proportion of demoulding breakage can be reduced, mostly permitting the total yields to be raised to 95-100%.

Substances suitable as plasticizers generally have a molecular weight of from 100 to 20,000 and contain one or more hydrophilic groups in the molecule, e.g. hydroxy groups, ester groups or amino groups. Suitable substances are citrates, phthalates, sebacates, castor oil. Examples of suitable plasticizers are alkyl citrates, glycerol esters, alkyl phthalates, alkyl sebacates, sucrose esters, sorbitan esters, dibutyl sebacate and polyethylene glycols 4000 to 20,000. Preferred plasticizers are tributyl citrate, triethyl citrate, triethyl acetylcitrate, dibutyl sebacate and diethyl sebacate. The amounts used are from 10 to 25, preferably from 12 to 22, particularly preferably from to 18, %.-% [sic] by weight, based on the (meth)acrylate copolymer.

### Dryers (Adhesion Preventers):

Dryers may be present in the mixture alone or together with release agents. Dryers in the mixture have the following properties: they have large specific surface areas, are chemically inert, have good flow, and are fine particles. Due to these properties, they can advantageously be homogeneously distributed in melts and lower the tack of polymers which contain polar comonomers acting as functional groups. Dryers (adhesion preventers) can be added in an amount can in an amount [sic] of from 1 to 50% by weight, preferably from 10 to 40% by weight, based on the copolymer.

### Examples of Dryers are:

aluminium oxide, magnesium oxide, kaolin, talc, silica (Aerosils), barium sulphate, carbon black and cellulose.

### Release Agents (Mould-Release Agents)

Release agents (mould-release agents) may be present in the mixture alone or together with dryers. Release agents (mould-release agents) have to be added in an amount can in an amount [sic] of from 0.1 to 3, preferably from 0.2 to 2.5, % by weight, based on the copolymer.

In contrast to dryers, mould-release agents have the property of reducing the adhesive force between the [lacuna] mouldings and the mould surface in which the moulding is produced. This makes it possible to produce mouldings which do not exhibit break-up or geometrical deformation. Mould-release agents are mostly partially compatible or incompatible with the polymers in which they are particularly active. When the melt is injected into the mould cavity, the partial compatibility or incompatibility results in migration to the boundary in the transition between mould wall and moulding. In order that mould-release agents can migrate particularly advantageously, the melting point of the mould-release agent is to be below the processing temperature of the polymer by from 20° C. to 100° C.

Examples of release agents (mould-release agents) are: esters of fatty acids or fatty amides, aliphatic, long-chain carboxylic acids, fatty alcohols and esters of these, montan waxes or paraffin waxes and metal soaps, and particular mention should be made of glycerol monostearate, stearyl alcohol, glycerol esters of behenic acid, cetyl alcohol, palmitic acid, stearic acid, canauba [sic] wax, beeswax, etc.

### Additives or Auxiliaries

The mixture may comprise from 0 to 100% by weight of auxiliaries or additives conventional in the pharmaceutical [lacuna], based on the (meth)acrylate copolymer.

Examples of those which may be mentioned here are stabilizers, dyes, antioxidants, wetting agents, pigments, gloss agents, etc. They serve primarily as processing aids and are intended [lacuna] reliable and reproducible production and good long-term storage stability can be ensured.

Examples of other auxiliaries for the purposes of the invention are polymers. The mixture may comprise from 0 to 20% by weight of another polymer or copolymer, based on [sic] the (meth)acrylate copolymer.

To control the release of active ingredient, it can be advantageous in a particular case to admix other polymers. The proportion of other polymers in the mixture is, however, not more than 20% by weight, preferably not more than 10% by weight, in particular from 0 to 5%.-% [sic] by weight, based on the (meth)acrylate copolymer.

Examples of these other polymers are: polyvinyl-pyrolidones [sic], polyvinyl alcohols, cationic (meth)acrylate copolymers made from methyl methacrylate and/or ethyl acrylate and 2-dimethylaminoethyl methacrylate (EUDRAGIT® E100), carboxymethylcellulose salts, hydroxypropylcellulose (HPMC), neutral (meth)acrylate copolymers made from methyl methacrylate and ethyl acrylate (dry matter from EUDRAGIT® NE 30 D), copolymers made from methyl methacrylate and butyl methacrylate (PLASTOID® B).

Anionic (meth)acrylate copolymers composed of from 40 to 100, preferably from 45 to 99, in particular from 85 to 95, % by weight of C₁-C₄-alkyl (meth)acrylates capable of free-radical polymerization and up to 60, preferably from 1 to 55, in particular from 5 to 15, % by weight of (meth)acrylate monomers having an anionic group in the alkyl radical are also suitable.

Examples of suitable materials are neutral (meth)acrylate copolymers made from 20 to 40% by weight of ethyl acrylate and from 60 to 80% by weight of methyl methacrylate (EUDRAGIT® NE).

Other suitable materials are anionic (meth)acrylate copolymers made from 40 to 60% by weight of methacrylic acid and from 60 to 40% by weight of methyl methacrylate, or from 60 to 40% by weight of ethyl acrylate (EUDRAGIT® L or EUDRAGIT® L100-55).

Other suitable materials are anionic (meth)acrylate copolymers made from 20-40% by weight of methacrylic acid and from 80 to 60% by weight of methyl methacrylate (EUDRAGIT® S).

Materials with particularly good suitability are (meth)acrylate copolymers composed of from 10 to 30% by weight of methyl methacrylate, from 50 to 70% by weight of methyl acrylate and from 5 to 15% by weight of methacrylic acid (EUDRAGIT® FS).

### Active Pharmaceutical Ingredient

The mixture may comprise from 0 to 200% by weight of one or more active pharmaceutical ingredients, based on the (meth)acrylate copolymer.

The active pharmaceutical ingredients to be used here comprise those which do not decompose at the processing temperature.

Drugs (active pharmaceutical ingredients) used for the purposes of the invention are intended for use on or in the human or animal body, in order to
1. cure, alleviate, prevent or detect diseases, suffering, bodily injury or pathological symptoms.
2. permit detection of the condition, the state, or the functions of the body, or of mental states.
3. replace body fluids or active materials produced by the human body or by the bodies of animals.
4. defend against, eliminate, or render harmless pathogens, parasites or exogenous substances, or
5. influence the condition, the state, or the functions of the body, or influence mental states.

Reference works, such as the Rote Liste or the Merck Index, should be referred to for commonly used drugs.

According to the invention use may be made of any active ingredient which complies with the desired therapeutic action in the sense of the definition above and which has sufficient stability or ability to penetrate the skin.

Without any claim to completeness, the following are important examples (classes and individual substances):

analgesics,
antiallergics, antiarrhythmics,
antibiotics, chemotherapeutics, antidiabetics, antidotes,
antiepileptics, antihypertensives, antihypotensives, anticoagulants, antimycotics, anti-inflammatory agents, beta-receptor blockers, calcium antagonists and ACE inhibitors,
broncholytics/antiasthmatics, cholinergics, corticoids (Interna),
dermatics, diuretics, enzyme inhibitors, enzyme preparations and transport proteins,
expectorants, geriatrics, gout remedies, influenza remedies,
hormones and their inhibitors, hypnotics/sedatives, cardiac stimulants, lipid-lowering agents, parathyroid hormones/calcium metabolism regulators, psychopharmaceuticals, sex hormones and their inhibitors,
spasmolytics, sympatholytics, sympathomimetics, vitamins,
wound treatment agents, cytostatics.

### Step b) of the Process

Prior to processing, the (meth)acrylate copolymer practically always has a content above 1% by weight, mostly around 2% by weight, of low-boiling constituents with a vapour pressure of at least 1.9 bar at 120° C. The low-boiling constituents are mainly water absorbed from atmospheric moisture.

Step b) of the process relates to the devolatilization of the mixture from step a) of the process at temperatures of at least 120° C., preferably 125 to 155° C., particularly preferably from 130 to 140° C., thereby lowering the content of the low-boiling constituents with a vapour pressure of at least 1.9 bar at 120° C. to not more than 0.5, preferably not more than 0.2, % by weight, particularly preferably not more than 0.1% by weight. This avoids any occurrence during step c) of the injection moulding process of undesirable sudden devolatilization which would form bubbles or cause foaming within the resultant moulding, which would then be unusable.

Since the (meth)acrylate copolymer has a glass transition temperature in the region of 50° C., low-boiling constituents cannot be removed by simple high-temperature drying, which would cause undesirable sintering or filming of the copolymer during the process.

For this reason, the devolatilizing step b) is preferably takes place [sic] via extrusion drying in an extruder with a devolatilization section, or via devolatilization in an injection moulding system with an upstream vent. In the case of the [lacuna] via extrusion drying in an extruder with a devolatilization section, the devolatilized extrudate is introduced immediately into the injection moulding machine, or into the injection mould. In the case of devolatilization in an injection moulding system with an upstream vent, the devolatilization takes place in an antechamber prior to the injection of the polymer melt into the injection mould.

The mixture may either be introduced immediately in melt form into an injection moulding system, or preferably first be cooled and pelletized. The pellets should be stored under conditions which permit little reabsorption of water, i.e. only for a short time and/or under dry storage conditions.

### Step c) of the Process

Injection of the devolatilized mixture at a temperature of from 80 to 160° C., preferably from 90 to 150° C., particularly preferably from 115 to 145° C., into the mould of an injection moulding system and removal of the resulting moulding from the mould. The temperature given indicates the maximum temperature reached in the hottest section of the injection moulding system used.

The thermoplastic processing takes place in a manner known per se by means of an injection moulding machine at temperatures in the range from 80 to 160° C., in particular from 100° C. to 150° C., and at pressures of from 60 to 400 bar, preferably from 80 bar to 120 bar.

If the glass transition temperatures of the (meth)acrylate copolymers used are in the range of, for example, from 40° C. to 60° C., the mould temperature is correspondingly lower, e.g. not more than 30 or not more than 20° C., so that the mixture present solidifies within the mould just a short time after the injection procedure, with the result that the finished moulding can be removed or demoulded.

The mouldings can be removed from the mould cavity of the injection mould without breakage and have a uniform, compact and defect-free surface. The moulding features mechanical strength and, respectively, elasticity and breaking strength.

In particular, it has an impact strength to ISO 179, measured on test specimens, of at least 15 KJ/m² [sic], preferably at least 18 KJ/m² [sic], particularly preferably at least 20 KJ/m2 [sic].

The VST (A10) approximate heat distortion temperature measured on test specimens to ISO 306 is from 30° C. to 60° C.

The mouldings obtained according to the invention may, for example, have the form of a capsule, have [sic] part of a capsule, e.g. of a half of a capsule, or of a hard capsule, these serving as a container for an active pharmaceutical ingredient. An example of a possible filling is active ingredients present in binders in the form of pellets, and the two parts of the capsule are then joined by adhesive bonding, laser-welding, ultrasound-welding, or microwave-welding, or by means of a snap-action connection.

According to the invention, capsules made from different material (e.g. gelatine, partially hydrolysed starch, HPMC or other methacrylates) can also be combined with one another by this process. The moulding can therefore also be a part of a dosage unit.

Other forms, such as tablets shapes or lenticular shapes, are also possible. The compounded material used for injection moulding here already comprises the active pharmaceutical ingredient. In the final form, the active ingredient is present with maximum uniformity of distribution in crystalline (solid dispersion) or dissolved amorphous form (solid solution).

### Mouldings

Due to step b) of the process, the injection mouldings obtained in step c) of the process have very low water content, at least immediately after production. The water content measurable by the “Karl Fischer” method on test specimens is in the range below 0.5% by weight. Subsequent changes in water content, for example through relatively long storage of the mouldings in a moist atmosphere, are beyond the relevance limits for the invention, since a low content of low-boiling constituents with a vapour pressure of at least 1.9 bar at 120° C., primarily water, is required primarily for the smooth working of step c) of the process.

A measure of the quality of the moulding obtained is what is known as the alkali value. The definition of the alkali value is similar to that of the acid value. It states how many mg potassium hydroxide (KOH) are equivalent to the basic groups in 1 g of polymer. It is determined by potentiometric titration as in Ph.Eur.2.2.20 “Potentiometric Titration” or USP<541>. The starting weight is an amount which corresponds to 1 g of a copolymer having 10% by weight of trimethylammoniumethly methacrylate [sic] chloride, and this is dissolved in a mixture of 96 ml of glacial acetic acid and 4 ml of purified water and titrated with 0.1 N perchloric acid against mercuric acetate (addition of 5 ml of a 5% strength solution in glacial acetic acid). The alkali value of a thermally degraded polymer in the mixture falls in comparison with the [lacuna] of a mixture with no thermal degradation.

Even small differences in the alkali value as small as 0.5 can indicate thermal degradation if they exceed 0.5. If this type of degradation is present there is a risk that the delayed-release properties have been altered unacceptably.

The process of the invention can give injection mouldings which can directly comprise an active pharmaceutical ingredient or which, e.g. in the form of a capsule, can enclose a subsequent filling of an active pharmaceutical ingredient.

Examples of active ingredients which are suitable fillings for the mouldings (capsules) or else are suitable for incorporation into the mouldings are: acetylsalicylic acid, ranitidine, simvastatin, enalapril, fluoxetine, amlodipine, amoxicillin, sertaline [sic], nifidipine [sic], ciprofloxacin, acycolvir [sic], lovastatin, epoetin, paroxetine, captopril, nabumetone, granisetron, cimetidine, ticarcillin, triamterene, hydrochlorothiazide, verapamil, paracetamol, morphine derivatives, topotecan or of [sic] the salts used pharmaceutically.

The formulation of the invention is suitable for administration of, in principle, any desired active pharmaceutical ingredients which are preferably intended to be released in the intestine and/or colon, in particular those which can advantageously be administered in delayed-release form, e.g. antidiabetics, analgesics, anti-inflammatory agents, antirheumatic agents, antihypotensives, antihypertensives, psycho-pharmaceuticals, tranquillizers, antiemetics, muscle relaxants, glucocorticoids, agents for treating ulcerative colitis or Crohn's disease, antiallergics, antibiotics, antiepileptics, anticoagulants, antimycotics, antitussives, arteriosclerosis remedies, diuretics, enzymes, enzyme inhibitors, gout remedies, hormones and their inhibitors, cardiac glycosides, immunotherapeutics and cytokines, laxatives, lipid-lowering agents, migraine remedies, mineral preparations, otologicals, anti-Parkinson agents, thyroid therapeutics, spasmolytics, platelet aggregation inhibitors, vitamins, cytostatics and metastasis inhibitors, phytopharmaceuticals, chemotherapeutics and amino acids.

Examples of suitable active ingredients are acarbose, beta-receptor blockers, non-steroidal anti-rheumatic agents, cardiac glycosides, acetylsalicylic acid, virustatics, aclarubicin, acyclovir, cisplatin, actinomycin, alpha- and beta-sympatomimetics, (dmeprazole [sic], allopurinol, alprostadil, prostaglandins, amantadine, ambroxol, amlodipine, methotrexate, S-aminosalicylic [sic] acid, amitryptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cephalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglycic acid, coumarin and coumarin derivatives, cysteine, cytarabine, cyclophosphamide, cyclosporin, cyproterone, cytarabine, dapiprazole, desogestrel, desonide, dihydralazine, diltiazem, ergot alkaloids, dimenhydrinate, dimethyl sulphoxide, dimethicone, dipyridarnoi [sic], domperidone and domperidane [sic] derivatives, dopamine, doxazosine, doxorubicin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenyloin, riluzoles, risedronate, sildenafil, topiramate, macrolide antibiotics, oestrogen and oestrogen derivatives, gestagen and gestagen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, flunarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, St John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramin, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixen, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novaminsulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocin, pentifylline, pentoxifylline, perphenazine, pethidine, plant extracts, phenazone, pheniramine, barbituric acid derivatives, phenylbutazone, phenyloin, pimozide, pindolol, piperazine, piracetam, pirenzepine, piribedil, piroxicam, pramipexol, pravastatin, prazosin, procaine, promazine, propiverine, propranolol, propyphenazone, prostaglandins, protionamide, proxyphylline, quetiapine, quinapril, quinaprilate, ramipril, ranitidine, reproterol, reserpine, ribavarin, rifampicin, risperidone, ritonavir, ropinirol, roxatidine, roxithromycin, ruscogenin, rutoside and rutoside derivatives, sabadilla, salbutamol, salmeterol, scopolamine, selegiline, sertaconazole, sertindol, sertralione [sic], silicates, simvastatin, sitosterol, sotalol, spaglumic acid, sparfloxacin, spectinomycin, spiramycin, spirapril, spironolactone, stavudine, streptomycin, sucralfate, sufentanil, sulbactam, sulfonamides, sulfasalazine, sulpiride, sultamicillin, sultiam, sumatriptan, suxamethonium chloride, tacrine, tacrolimus, taliolol, tamoxifen, taurolidine, tazaroten, temazepam, teniposide, tenoxicam, terazosin, terbinafine, terbutaline, terfenadine, terlipressin, tertatolol, tetracyclines, tetryzoline, theobromine, theophylline, butizine, thiamazol, phenothiazines, thiotepa, tiagabine, tiapride, priopionic acid derivatives, ticlopidine, timolol, tinidazole, tioconazole, tioguanine, tioxolone, tiropramide, tizanidine, tolazoline, tolbutamide, tolcapone, tolnaftate, tolperisone, topotecan, torasemide, anti6strogens [sic], tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutin, tulobuterol, tyramine, tyrothricin, urapidil, ursodeoxycholic acid, chenodeoxycholic acid, valaciclocir, valproic acid, vancomycin, vecuronium chloride, viagra, venlafaxine, verapamil, vidarabine, vigabatrin, viloxazine, vinblastine, vincamine, vincristine, visdesine, vinorelbine, vinpocetine, viquidil, warfarin, xantinol nicotinate, xipamide, zafirlukast, zalcitabine, zidovudine, zolmitriptan, zolpidem, zoplicone, zotepine and the like.

Examples of particularly preferred active ingredients are analgesics, such as tramadol or morphine, agents for treating ulcerative colitis or Crohn's disease, such as 5-aminosalicylic acid, corticosteroids, such as budesonide, proton pump inhibitors, such as omeprazole, virusstatics, such as acyclovir, lipid-lowering agents, such as simvastatin or pravastatin, H2 blockers, such as ranitidine or famotidine, antibiotics, such as amoxicillin and/or clavulanic acid, and ACE inhibitors, such as enalapril or amlodipine.

Where desired, the active ingredients may also be used in the form of their pharmaceutically acceptable salts or derivatives, and in the case of chiral active ingredients it is possible to use either optically active isomers or else racemates or diastereoisomer mixtures. If desired, the compositions of the invention may also comprise two or more active pharmaceutical ingredients.

### EXAMPLES

### Comparative Example 1

### Temperature Too High

### Devolatilization and Preparation of the Mixture (Compounded Material)

3.25 kg of EUDRAGIT® RL 100 pellets and 1.0 kg of talc are weighed into a 10 l stainless steel mixing container and then mixed for 5 min on a tumbling mixer.

The mixture prepared was fed to a 30.34 twin-screw extruder (Leistritz) to prepare a compounded material of the invention. The melt temperature measured was 140° C. and the screw rotation rate was 120 rpm. At a point downstream of 50% of the total length of the extruder screw, triethyl citrate plasticizer was added through an aperture in the barrel wall by way of a membrane pump, its amount being 15%, based on the copolymer. Downstream of a mixing section for homogenizing the mixture, it was devolatilized by way of a vent in the extruder barrel. Four extrudates were shaped by means of the die at the end of the extruder, and drawn off by way of a cooled metal plate and chopped to give pellets. A water content of 0.09% by weight was determined on the resultant pellets by means of Karl Fischer titration.

### Injection Moulding

The resultant mixture (compounded material) was fed to the hopper of an injection moulding machine (Arburg Allrounder 250-125), and the mouldings were injection moulded. The following temperatures were set on the injection moulding machine: section 1 (feed section): 70° C., section 2: 120° C., section 3: 160° C., section 4: 160° C., section 5 (die): 130° C. Injection pressure: 60 bar, cold pressure: 50 bar, back pressure: 5 bar. Mould temperature: 17° C. (cooled)

The moulding injection moulded was a 65×40×1 mm plaque.

Plaques free from streaks could be produced with a defect-free smooth surface. The plaques could be demoulded without difficulty and are geometrically stable. However, degradation of the polymer is to be expected, due to the high temperature.

### Comparative Example 2

### No Plasticizer

### Devolatilization

Preparation takes place as in Example 1, but without adding triethyl citrate plasticizer.

### Injection Moulding

Took place as described in Example 1. In section 3 and section 4 temperatures of 120° C. were set.

Result: It was not possible to produce uniform mouldings of correct geometrical shape. The cause lies in the excessively low flowability of the EUDRAGIT® RL 100 polymer.

### Example 3

### Inventive

### Devolatilization and Preparation of the Compounded Material

Preparation takes place as in Example 1.

### Injection Moulding

The resultant mixture (compounded material) was fed to the hopper of an injection moulding machine (Arburg Allrounder 250-125) and mouldings were injection moulded. However, in section 3 and section 4 of the injection moulding machine temperatures of 120° C. were set. The moulding injection moulded was a 65×40×1 mm plaque.

Plaques free from streaks could be produced with a defect-free smooth surface. The plaques could be demoulded without difficulty and are geometrically stable.

The alkali value of the resultant mouldings was determined. The definition of the alkali value is similar to that of the acid value. It states how many mg potassium hydroxide (KOH) are equivalent to the basic groups in 1 g of polymer. It is determined by potentiometric titration as in Ph.Eur.2.2.20 “Potentiometric Titration” or USP<541>. The starting weight is an amount which corresponds to 1 g of EUDRAGIT® RL 100, and is dissolved in a mixture of 96 ml of glacial acetic acid and 4 ml of purified water and titrated with 0.1 N perchloric acid against mercuric acetate (addition of 5 ml of a 5% strength solution in glacial acetic acid). The resultant alkali value obtained (mg KOH/g of polymer) was 23.1. In a comparison with a EUDRAGIT® RL 100 polymer not thermally stressed by the injection moulding process, the result is comparably good, with an alkali value of 22.9.

### Comparative Example 4

### No Dryer or Mould-Release Agent

### Devolatilization and Preparation of the Compounded Material

Gravimetric metering equipment was used to meter 10 kg of EUDRAGIT® RL 100 per hour into the feed section of the twin-screw extruder. Using a screw rotation rate of 120 rpm, the pellets were drawn into the extruder and plastified. The melt temperature set was 140° C.

At a point downstream of 50% of the total length of the twin-screw extruder, an aperture has been made in the barrel wall, and is used to introduce triethyl citrate by means of a membrane pump, its amount being 20%, based on the amount of polymer.

Downstream of a mixing section for homogenizing the mixture, devolatilization took place via another aperture in the barrel wall. Four extrudates were shaped by means of the die at the end of the extruder, and drawn off by way of a cooled metal plate and chopped to give pellets. A water content of 0.1% was determined on the resultant pellets by means of Karl Fischer titration.

### Injection Moulding

The resultant mixture (compounded material) was fed to the hopper of an injection moulding machine (Arburg Allrounder 250-125) and mouldings were injection moulded. However, in section 3 and section 4 of the injection moulding machine, temperatures of 140° C. were set. The moulding injection moulded was a 65×40×1 mm plaque.

The moulding injection moulded was a 65×40×1 mm plaque [sic].

After as little as two shots, the mouldings were observed to have increased tack and separation from the mould was observed to become more difficult, with the result that the experiment had to be terminated.

### Comparative Example 5

### Temperature Too High

### Devolatilization and Preparation of the Compounded Material

From devolatilized compounded material as in Example 1 comprising EUDRAGIT® RL 100.

### Injection Moulding

The resultant mixture (compounded material) was fed to the hopper of an injection moulding machine (Arburg Allrounder 250-125) and mouldings were injection moulded. However, in section 3 and section 4 of the injection moulding machine, temperatures of 170° C. were set. The moulding injection moulded was a 65×40×1 mm plaque.

Plaques free from streaks could be produced with a defect-free smooth surface. The plaques could be demoulded without difficulty and are geometrically stable.

The alkali number was determined on the resultant mouldings by means of potentiometry, using the method described in Example 3.

The result obtained was an alkali value (mg KOH/g of polymer) of 22.3. For comparative purposes, a EUDRAGIT® RL 100 polymer not subjected to the thermal stress of the injection moulding process was tested. The result obtained was an alkali value of 22.9. Although the value is close to the limit of analytical accuracy, [lacuna] indicate the problems of thermal decomposition above 160° C., [sic] Even at this temperature, marked degradation is to be expected, in particular during continuous operation

### Example 6

### Inventive

### Devolatilization and Preparation of the Compounded Material

3.25 kg of EUDRAGIT® RL 100 pellets and 1.0 kg of talc are weighed into a 10 l stainless steel mixing container and then mixed for 5 min on a tumbling mixer.

The mixture prepared was fed to a 30.34 twin-screw extruder (Leistritz) to prepare a compounded material of the invention. The melt temperature set was 140° C. and the screw rotation rate was 120 rpm. At a point downstream of 50% of the total length of the extruder screw, triethyl citrate plasticizer was added through an aperture in the barrel wall by way of a membrane pump, its amount being 20%, based on the total amount of material. Downstream of a mixing section for homogenizing the mixture, it was devolatilized by way of another aperture in the barrel wall. Four extrudates were shaped by means of the die at the end of the extruder, and drawn off by way of a cooled metal plate and chopped to give pellets. A water content of less than 0.1% was determined on the resultant pellets by means of Karl Fischer titration.

### Injection Moulding

The resultant mixture (compounded material) was fed to the hopper of an injection moulding machine (Arburg Allrounder 250-125), and the mouldings were injection moulded. However, in section 3 and section 4 of the injection moulding machine, temperatures of 140° C. were set. The moulding injection moulded was a 65×40 1 mm plaque.

The moulding injection moulded was a 65×40×1 mm plaque [sic].

Plaques free from streaks could be produced with a defect-free smooth surface. The plaques could be demoulded without difficulty and are geometrically stable.

### Example 7

### Inventive

### Devolatilization and Preparation of the Compounded Material

3.25 kg of EUDRAGIT® RL 100 pellets and 3.25 kg of EUDRAGIT® RS 100 pellets and 0.03 kg of stearic acid are weighed into a 10 l stainless steel mixing container and then mixed for 5 min on a tumbling mixer.

The mixture prepared was fed to a 30.34 twin-screw extruder (Leistritz) to prepare a compounded material of the invention. The melt temperature set was 140° C. and the screw rotation rate was 120 rpm. At a point downstream of 50% of the total length of the extruder screw, triethyl citrate plasticizer was added through an aperture in the barrel wall by way of a membrane pump, its amount being 10%, based on the total amount of material. Downstream of a mixing section for homogenizing the mixture, it was devolatilized by way of another aperture in the barrel wall. Four extrudates were shaped by means of the die at the end of the extruder, and drawn off by way of a cooled metal plate and chopped to give pellets. A water content of 0.15% was determined on the resultant pellets by means of Karl Fischer titration.

### Injection Moulding

The resultant mixture (compounded material) was fed to the hopper of an injection moulding machine (Arburg Allrounder 250-125), and the mouldings were injection moulded. However, in section 3 and section 4 of the injection moulding machine, temperatures of 140° C. were set. The moulding injection moulded was a 65×40×1 mm plaque.

The moulding injection moulded was a 65×40×1 mm plaque [sic].

Plaques free from streaks could be produced with a defect-free smooth surface. The plaques could be demoulded without difficulty and are geometrically stable

### Example 8

### Inventive

### Devolatilization and Preparation of the Compounded Material

3.25 kg of EUDRAGIT® RL 100 pellets and 0.01 kg of stearic acid are weighed into a 10 l stainless steel mixing container and then mixed for 5 min on a tumbling mixer.

The mixture prepared was fed to a 30.34 twin-screw extruder (Leistritz) to prepare a compounded material of the invention. The melt temperature set was 140° C. and the screw rotation rate was 120 rpm. At a point downstream of 50% of the total length of the extruder screw, triethyl citrate plasticizer was added through an aperture in the barrel wall by way of a membrane pump, its amount being 12.5%, based on the total amount of material. Downstream of a mixing section for homogenizing the mixture, it was devolatilized by way of another aperture in the barrel wall. Four extrudates were shaped by means of the die at the end of the extruder, and drawn off by way of a cooled metal plate and chopped to give pellets. A water content of 0.13% was determined on the resultant pellets by means of Karl Fischer titration.

### Injection Moulding

The resultant mixture (compounded material) was fed to the hopper of an injection moulding machine (Arburg Allrounder 250-125), and the mouldings were injection moulded. However, in section 3 and section 4 of the injection moulding machine, temperatures of 140° C. were set. The moulding injection moulded was a 65×40×1 mm plaque.

Plaques free from streaks could be produced with a defect-free smooth surface. The plaques could be demoulded without difficulty and are geometrically stable.

### Example 9

### Inventive

### Devolatilization and Preparation of the Compounded Material

3.25 kg of EUDRAGIT® RS 100 pellets and 0.003 kg of stearic acid are weighed into a 10 l stainless steel mixing container and then mixed for 5 min on a tumbling mixer.

The mixture prepared was fed to a 30.34 twin-screw extruder (Leistritz) to prepare a compounded material of the invention. The melt temperature set was 140° C. and the screw rotation rate was 120 rpm. At a point downstream of 50% of the total length of the extruder screw, triethyl citrate plasticizer was added through an aperture in the barrel wall by way of a membrane pump, its amount being 10%, based on the total amount of material. Downstream of a mixing section for homogenizing the mixture, it was devolatilized by way of another aperture in the barrel wall. Four extrudates were shaped by means of the die at the end of the extruder, and drawn off by way of a cooled metal plate and chopped to give pellets. A water content of 0.04% was determined on the resultant pellets by means of Karl Fischer titration.

### Injection Moulding

The resultant mixture (compounded material) was fed to the hopper of an injection moulding machine (Arburg Allrounder 250-125), and the mouldings were injection moulded. However, in section 3 and section 4 of the injection moulding machine, temperatures of 140° C. were set. The moulding injection moulded was a 65×40×1 mm plaque.

Plaques free from streaks could be produced with a defect-free smooth surface. The plaques could be demoulded without difficulty and are geometrically stable.

What is claimed is:

1. An injection molded product selected from a capsule shell or other dosage unit composed of a composition comprising:
a) a (meth)acrylate copolymer composed of from 85 to 98% by weight of C1-C4-alkyl (meth)acrylates capable of free-radical polymerization and from 15 to 2% by weight of (meth)acrylate monomers having a quaternary ammonium group in the alkyl radical;
b) a plasticizer present from 10 to 25% by weight;
c) a release agent present from 0.1 to 3% by weight;
d) a drying agent present from 10 to 50% by weight;
e) optionally a first pharmaceutical active agent present from 0 to 100% by weight; and
f) optionally a pharmaceutically acceptable additive or auxiliary; the amounts of the active agent, plasticizer, drying agent and release agent based on the (meth)acrylate copolymer.

2. The molded product according to claim 1, wherein the plasticizer is an alkyl citrate, a glycerol ester, an alkyl phthalate, an alkyl sebacate, a sucrose ester, a sorbitan ester, dibutyl sebacate or a polyethylene glycol 4000 to polyethylene glycol 20,000.

3. The molded product according to claim 2, wherein the plasticizer is tributyl citrate, triethyl citrate, triethyl acetylcitrate, dibutyl sebacate or diethyl sebacate.

4. The molded product according to claim 3, wherein the composition comprises from 12 to 22% by weight of the plasticizer, based on the (meth)acrylate copolymer.

5. The molded product according to claim 1, wherein the release agent is an ester of a fatty acid or a fatty amides, an aliphatic long-chain carboxylic acid, a fatty alcohol or esters thereof, a montan wax or a paraffin wax.

6. The molded product according to claim 1, wherein the release agent is glycerol monostearate, stearyl alcohol, glycerol esters of behenic acid, cetyl alcohol, palmitic acid, stearic acid, canauba wax, or beeswax.

7. The molded product according to claim 6, wherein the release agent is stearyl alcohol.

8. The molded product according to claim 1, wherein the C1-C4-alkyl (meth)acrylate is methyl acrylate, ethyl acrylate, butyl acrylate, butyl methacrylate and methyl methacrylate.

9. The molded product according to claim 1, wherein the (meth)acrylate monomer is 2-trimethylammoniumethyl methacrylate chloride.

10. The molded product according to claim 1 wherein the copolymer is from 50-70% by weight of methyl methacrylate, from 20 to 40% by weight of ethyl acrylate and from 7 to 2% by weight of 2-trimethylammoniumethyl methacrylate chloride.

11. The molded product according to claim 1 wherein the copolymer is from 65% by weight of methyl methacrylate, 30% by weight of ethyl acrylate and 5% by weight of 2-trimethylammoniumethyl methacrylate chloride.

12. The molded product according to claim 1, wherein the drying agent is aluminium oxide, magnesium oxide, kaolin, talc, silica, barium sulphate, carbon black or cellulose.

13. The molded product according to claim 1 which further comprises a second polymer present from not more than 20% by weight.

14. The molded product according to claim 13 wherein the second polymer is polyvinyl-pyrolidone, polyvinyl alcohol, cationic (meth)acrylate copolymers made from methyl methacrylate and/or ethyl acrylate and 2-dimethylaminoethyl methacrylate, carboxymethylcellulose salts, hydroxypropylcellulose (HPMC), neutral (meth)acrylate copolymers made from methyl methacrylate and ethyl acrylate, or a copolymer made from methyl methacrylate and butyl methacrylate.

15. The molded product according to claim 1, wherein the first pharmaceutical active agent is present within the molded product and is selected from the group consisting of acetylsalicylic acid, rantidine, simvastatin, enalapril, fluoroxetine, amlodipine, amoxicillin, sertaline, nifidipine, ciprofloxacin, acycolvir, lovastatin, epoetin, paroxetine, captopril, nabumetone, granisetron, cimetidine, ticarcillin, triamterene, hydrochlorothiazide, verapamil, paracetamol, morphine derivatives, topotecan, or pharmaceutical salts thereof.

16. The molded product according to claim 15, wherein the first pharmaceutical active agent undergo a delayed release in the gastrointestinal tract of an animal or human from said molded product.

17. The molded product according to claim 1, which has an impact strength according to ISO 179 of at least 15 KJ/m2.
